(19) United States
(12) Patent Application Publication
(10) Pub. No.: US 2026/0033993 A1
(43) Pub. Date: Feb. 5, 2026

(54) SKIN-ADHESIVE AIR-PERMEABLE INTELLIGENT BANDAGE

(71) Applicant: City University of Hong Kong, Hong Kong (HK)

(72) Inventors: Xinge YU, Hong Kong (HK); Xingcan HUANG, Hong Kong (HK); Qiang ZHANG, Hong Kong (HK); Yawen YANG, Hong Kong (HK); Lung CHOW, Hong Kong (HK)

(21) Appl. No.: 19/287,754

(22) Filed: Jul. 31, 2025

Related U.S. Application Data
(60) Provisional application No. 63/678,566, filed on Aug. 2, 2024.

(51) Int. Cl. A61F 13/02; A61F 13/00; A61L 15/18; A61L 15/24; A61L 15/32; A61L 15/42; A61M 35/00; A61N 1/04; D04H 1/4291; D04H 1/4382; D04H 1/728; D06M 10/00; D06M 10/08; D06M 11/83; D06M 13/238; D06M 101/20; D06M 101/26

(52) U.S. Cl. CPC A61F 13/023; A61F 13/00063; A61F 13/0276; A61L 15/18; A61L 15/24; A61L 15/32; A61L 15/42; A61M 35/10; A61N 1/0428; D04H 1/4291; D04H 1/43835; D04H 1/728; D06M 10/001; D06M 10/08; D06M 11/83; D06M 13/238; A61M 2205/0205; A61M 2205/0216; A61M 2205/0233; A61M 2230/201; A61M 2230/208; A61M 2230/50; D06M 2101/20; D06M 2101/26; D10B 2321/08; D10B 2321/121; D10B 2509/022

(57) ABSTRACT

The present invention provides a skin-adhesive air-permeable intelligent bandage comprising: a stretchable adhesive antibacterial bioelectrical interface film made of stretchable adhesive antibacterial fibres; a waterproof moisture-permeable protective film for protecting the wound from external contaminants; and a permeable stretchable circuit assembly arranged between the bioelectrical interface film and the protective film. The permeable stretchable circuit assembly comprises: a permeable stretchable circuit board; one or more biosensors constructed on the permeable stretchable circuit board; and electronic components assembled on the permeable stretchable circuit board. The electronic components include: a physiological signal processing module electrically coupled to the one or more biosensors for in-situ wound monitoring; and a drug delivery actuation module electrically coupled to the bioelectrical interface film for adaptive drug delivery for wound treatment. The provided bandage is more convenient, comfortable and efficient without numerous dressings, thereby not hindering the daily activities and life quality of patients.

### CROSS-REFERENCE TO RELATED APPLICATIONS

The present application claims priority from the U.S. Provisional Patent Application No. 63/678,566 filed on 2 Aug. 2024, and the disclosure of which is incorporated herein by reference in its entirety.

### FIELD OF THE INVENTION

The present invention generally relates to intelligent wound management technology. More specifically the present invention relates to an intelligent skin-adhesive, highly integrated permeable electronic bandage.

### BACKGROUND OF THE INVENTION

Chronic wounds, always caused by diabetic ulcers, nonhealing surgical incisions, and venous ulceration, frequently present with severe bacterial infection and impaired healing, which would lead to unbearable pain for patients and huge financial burdens. The healing of chronic wounds would go through several phrases of hemostasis, inflammation, new tissue formation and remodeling, and each healing phrase may be influenced by multiple environmental and physiological factors. Typically, these factors and the state of wound healing can be inferred from the dynamic changes in wound exudates. For example, research has suggested that wound healing is accompanied by a reduction in exudate pH levels from alkaline to neutral, and a rise in the skin temperature around an acute wound during the initial days post-infection. Besides, monitoring glucose concentration in wound exudates facilitates the regulation of blood glucose in diabetic patients, thereby reducing infection risks.

Clinically, various wound dressings (like gauze dressing, foam dressing, alginate dressing) are always prepared to serve multifaceted needs, including absorbing excess wound exudate, keeping moist environments, preventing wound infection and wearable conformability et al. Although these wound dressings are able to meet various types of wounds, it is inevitable to combine multiple wound dressings for complex wounds, which would largely increase the medical costs and healing periods. Besides, patients with severe open sores (such as venous ulcers, diabetic ulcers, and pressure sores) may not respond to traditional treatments, requiring further intervention like skin grafting surgery to achieve wound healing. Current digital wound dressings have integrated with wireless communications and biosensors, but they always have large sizes, poor mechanical properties and stretchability with rigid batteries. Although these commonly used batteries, like lithium-ion batteries, can provide sufficient power sources for remote communication and data processing, rigid encapsulation and potential safety risks have limited their applications in epidermal, soft and bio-integrated electronics. Most digital wound dressings have a single-function mechanism, either monitoring or pro-healing. These effects are commonly passive, incapable of responding to or adjusting according to the dynamic changes of wound microenvironments.

### SUMMARY OF THE INVENTION

To address abovesaid requirements, the present invention provides an ultrathin, battery-free, skin-adhesive, permeable, waterproof, stretchable, antibacterial and multifunctional electronics intelligent wound management system capable of in-situ wound monitoring, accelerating wound healing and intelligent wound treatment. The intelligent wound management system provided by the present invention is ultrathin with a similar thickness to the epidermis and excellent skin-adhesion, capable of conforming to skin deformation. The intelligent wound management system not only shows the same functions as human skin in terms of permeability, stretchability, and adaptability but also encompasses properties including antibacterial ability, hemostasis, water retention, and pro-healing. With wireless communication and powering based on near-field communication (NFC), the battery-free intelligent wound management system can real-time and in-situ monitor the wound conditions by assessing the glucose level, pH and temperature (Tem) in wound exudate combined with the stretchable state-of-the-art biosensors, and thus simultaneously adjusting the treatment for avoiding the wound infection. Extensive in vitro and in vivo evaluations illustrate the ability of the intelligent wound management system to monitor wound conditions and accelerate wound healing.

Leveraging the cutting-edge bioelectronic integration strategy, NFC technology and microelectronic fabrication methods, the intelligent wound management system is able to be adhered to wound location and achieve wireless communication and data transfer with a smart terminal (e.g., smartphones) without the need for any battery supplies. The bioelectrical interface film integrates essential bioactive properties, including biocompatibility, skin adherence, antibacterial action, hemostatic capabilities, gas permeability, and waterproofing, all of which passively enhance wound healing and infection prevention. The biosensors perform in-situ monitoring of glucose, pH, and temperature of wound beds, providing critical insights into the wound conditions. At the same time, the smart terminal can supply power for the intelligent wound management system for intelligent adaptive and on-demand drug release and wound treatment when wound infection occurs.

When infection occurs at the wound beds, the intelligent wound management system could achieve the adaptive drug delivery and on-demand treatment by assessing the wound infection status. Besides, the intelligent wound management system incorporates salient features including permeability, adaptability, biocompatibility, skin-adhesion, hemostasis, antibacterial and waterproof properties for preventing wound infection and improving the wearing comfortability, water retention, hydrophilicity and accelerating cell migration properties for maintaining a moist wound environment and promoting wound healing

In accordance with the first aspect of the present invention, a skin-adhesive air-permeable intelligent bandage is provided. The bandage comprises: a stretchable adhesive antibacterial bioelectrical interface film made of stretchable adhesive antibacterial fibres; a waterproof moisture-permeable protective film for protecting the wound from external contaminants; and a permeable stretchable circuit assembly arranged between the bioelectrical interface film and the protective film. The permeable stretchable circuit assembly comprises: a permeable stretchable circuit board; one or more biosensors constructed on the permeable stretchable circuit board; and electronic components assembled on the permeable stretchable circuit board. The electronic components include: a physiological signal processing module electrically coupled to the one or more biosensors for in-situ wound monitoring; and a drug delivery actuation module electrically coupled to the bioelectrical interface film for adaptive drug delivery for wound treatment.

Preferably, the stretchable adhesive antibacterial bioelectrical interface is a fibrous film made by co-electrospinning of styrene-ethylene-butylene-styrene (SEBS) fibers and gelatin methacrylate (GelMA) fibers loaded with antibiotics.

Preferably, the weight ratio of the SEBS fibers to the GelMA fibers is equal to 1:1.

Preferably, the bioelectrical interface film is modified with tannic acid to contain catechol and pyrogallol, and adherable to the skin through multiple synergistic reactions including hydrogen bonding and Schiff base/Michael addition reactions, electrostatic attraction, and cation-π interactions.

Preferably, the fibrous film is loaded with electrically conductive nanoparticles.

Preferably, the electrically conductive nanoparticles are silver nanoparticles.

Preferably, the bandage further comprises a wireless energy harvesting and communication module for enabling the skin-adhesive air-permeable intelligent bandage to be in communication with a remote device to facilitate battery-free remote diagnosis and treatment of wound.

Preferably, the drug delivery actuation module includes: at least one pair of drug delivery electrodes for applying a drug delivering voltage on the bioelectrical interface film; and a switch connected to the drug delivery electrodes and configured for switching on/off the drug delivering voltage.

Preferably, the bandage further comprises a microcontroller unit electrically connected to the drug delivery actuation module and configured to adjust drug delivery amount by controlling a period of switching on the drug delivering voltage.

Preferably, the one or more biosensors include a glucose sensor having least one stretchable working electrode attached to the permeable stretchable circuit board; the working electrode has a layered-structure including: a stretchable base; a mediator layer deposited on the stretchable base; a layer of glucose sensing element deposited on the mediator layer; and a layer of entrapping material deposited on the layer of glucose sensing element.

Preferably, the one or more biosensors include a pH sensor having least one stretchable working electrode attached to the permeable stretchable circuit board; and the working electrode has a layered-structure including: a stretchable base; and a layer of pH sensing element deposited on the stretchable base.

Preferably, the one or more biosensors include a thermal sensor attached on the permeable stretchable circuit board.

In accordance with the second aspect of the present invention, a method for manufacturing the skin-adhesive air-permeable intelligent bandage of the first aspect is provided. The method comprises: fabricating the bioelectrical interface film; fabricating the stretchable circuit assembly; attaching the bioelectrical interface film on a bottom side of stretchable circuit assembly; and covering a top side of the stretchable circuit assembly with a protective film.

Preferably, the bioelectrical interface film is fabricated by: preparing a first syringe containing a SEBS precursor solution and a second syringe containing a drug-loaded GelMA solution; connecting the first and second syringes to a dual-channel syringe pump to eject out SEBS fiber and GelMA fiber simultaneously; co-electrospinning the SEBS fiber and GelMA fiber on a metallic foil covering on a rotating drum to form a fibrous film composed of SEBS and GelMA fibers; immersing the fibrous film in a solution of 2-Hydroxy-4′-(2-hydroxyethoxy)-2-methylpropiophenone under UV exposure to crosslink the GelMA fiber in the fibrous film; peeling off the fibrous film from the metallic foil; immersing the fibrous film in a silver nanoparticle solution to load silver nanoparticles to the fibrous film; and immersing the fibrous film in a tannic acid to form the bioelectrical interface film; the bioelectrical interface film being modified with tannic acid to contain catechol and pyrogallol, and adherable to the skin through multiple synergistic reactions including hydrogen bonding and Schiff base/Michael addition reactions, electrostatic attraction, and cation-π interactions.

Preferably, the stretchable circuit assembly is fabricated by: forming stretchable SEBS/Au electrodes; forming sensor electrodes based on the stretchable SEBS/Au electrodes; and bonding the sensor electrodes and electronic components on the stretchable circuit board. Preferably, the stretchable SEBS/Au electrodes are formed by: pre-stretching a SEBS film; depositing a first layer of Cr/Au on the SEBS film; releasing the SEBS film; depositing a second layer of Cr/Au on the first layer of Cr/Au to form a stretchable SEBS/Au layer; and patterning the stretchable SEBS/Au layer to form the stretchable SEBS/Au electrodes.

Compared to the conventional wound dressing methods, especially for complex wounds at joint locations, the application of the intelligent wound management system is more convenient, comfortable and efficient without bandage and numerous dressings, thereby not hindering the daily activities and life quality of patients.

Due to the skin-adhesive and ultrathin features, the intelligent wound management system can be seamlessly adhered to different body locations, especially the joint (like wrist) and bone (like scapula) locations and achieve conformable contact and arbitrary deformation with skin for the in-situ wound monitoring and adaptive treatment by sensor data acquisition, wireless communication and programmed voltage modulation with the NFC technology. As every component layer is stretchable, the intelligent wound management system can be bent, twisted and stretched to accommodate realistic body monitoring.

### BRIEF DESCRIPTION OF THE DRAWINGS

Embodiments of the invention are described in more details hereinafter with reference to the drawings, in which:

FIG. 1A shows a schematic diagram of an intelligent wound management (iSAFE) system (or intelligent adhesive bandage) in accordance with one embodiment of the present invention being adhered to a skin and operated by a remote device; and FIG. 1B shows a schematic diagram illustrating how drug is released through a bioactive interface film (SA²EF) to a wound bed.

FIG. 2 is a block diagram illustrating key components of the intelligent wound management system.

FIG. 3 shows an exploded-view illustration of the layered-structure of the intelligent wound management system.

FIGS. 4A to 4C shows layouts of stretchable conductive trace layers for the permeable stretchable circuit board in accordance with one embodiment.

FIG. 5 shows the electrode layout of working electrode (i.e., thermal, pH and glucose sensors), counter electrode (CE) and reference electrode (RE).

FIG. 6A to 6C show lay-by-layer structures of the working electrodes for glucose and pH sensors, the reference electrode, respectively.

FIG. 7 illustrate a flowchart of a method for fabricating the intelligent wound management system in accordance with one embodiment of the present invention.

FIG. 8 shows a schematic diagram illustrate a fabrication process flow for the bioelectrical interface film in accordance with one embodiment of the present invention.

FIG. 9A shows a frontal plane of the vertical integration approach for fabricating the stretchable circuit assembly. FIGS. 9B and 9C show a frontal plane and bottom view of the connection points between Cu traces and sensor electrodes.

FIG. 10 shows optical image of a prototype of stretchable circuit assembly.

FIG. 11 shows a schematic diagram for a fabrication process of stretchable electrodes for subsequent construction of biosensors.

FIG. 12 shows XRD patterns of the SEBS, GelMA, SEBS/GelMA and SA²EF electrospinning films;

FIG. 13 shows conductivities of SA²EFs with different ratios of SEBS and GelMA.

FIG. 14A shows stress-strain curves of the blend electrospinning films with different ratios SEBS and GelMA, and the optimized SA²EF; FIG. 14B shows the stress-strain curves of GelMA, S/G fiber of content ratio equal to 1:1.

FIG. 15A shows resistance responses of the BEWI subjected to 10%, 30% and 50% strain as a function of time; FIG. 15B shows time-dependent resistance responses of the BEWI under continuou 50% cyclic stretching over 600 repetitions;

FIG. 16A shows mass losses of the electrospinning SEBS, GelMA, SEBS/GelMA and SA²EF films; FIG. 16B shows water vapor transmission rate of the SEBS, GelMA, SEBS/GelMA and SA²EF electrospinning films; FIG. 16C shows water losses of the SEBS, GelMA, SEBS/GelMA and SA²EF electrospinning films; FIG. 16D shows schematic illustration of the skin adhesive principles of the SA²EF.

FIGS. 17A and 17B show optical images and schematics of the peeling and shearing tests; FIGS. 17C and 17D shows comparison of the peel-off strength and shear stress between the SA²EF and commercial fibrin glue.

FIG. 18 illustrates live/dead staining of NIH/3T3 cells cultured with control material, SEBS/GelMA, and SA²EF for 24, 48, and 72 hours.

FIG. 19A shows cell viability calculated based on live/dead staining images; FIG. 19B shows cell proliferation evaluation via CCK-8 test.

FIG. 20 shows scratch assay to analyze the migration ability of NIH/3T3 cells pretreated with SA²EF.

FIGS. 21A and 21B illustrate images of cultivated E. coli and S. aureus colonies onto LB agar plates after treated with different materials respectively.

FIGS. 22A and 22B illustrate quantitative evaluation of bacterial colony and anti-bacterial rate of various materials respectively.

FIG. 23A illustrates comparison of the resistance responses between the normal SEBS/Au electrode and the pre-stretched SEBS/Au electrode under different strain levels; FIG. 23B illustrate time-dependent resistance responses of the stretchable SEBS/Au electrode subjected to continuous 30% strain for 500 cycles.

FIG. 24A illustrates time-dependent resistance change rate of SEBS/Au electrode subjected to continuous friction by a glass rod; FIG. 24B illustrate resistance values of the SEBS/Au electrode as a function of friction cycles. The resistance value of the electrode could stay basically the same with that of initial state after continuous dynamic friction, demonstrating the stability of the conductive SEBS/Au electrode.

FIGS. 25A to 25C illustrate responses of the glucose, pH and temperature sensors as a function of time respectively.

FIGS. 26A to 26C illustrate corresponding calibration curves of the glucose, pH and temperature sensors.

FIG. 27A shows live/dead staining of NIH/3T3 cells cultured with pH, glucose and temperature sensors for 24, 48, and 72 hours; FIG. 27B illustrates cell viability calculated based on live/dead staining images; FIG. 27C illustrates cell proliferation evaluation via CCK-8 test.

FIG. 28A shows optical image of the iSAFE adhered on the rat with a chronic wound on back for in-situ wound monitoring and accelerating wound healing; FIG. 28B shows time-line of the in-vivo study on a wound infection model;

FIG. 28C shows optical images and schematic illustrations of the infected wound of diabetic rats with three different wound dressings; FIG. 28D shows relative wound size changes of diabetic rats with three different wound dressings as a function of days; FIGS. 28E to 28G show glucose concentration, pH and temperature in the wound exudate of diabetic rats with three different wound dressings as a function of days; FIG. 28H shows optical images of the colonies derived from infected wound of diabetic rats; FIGS. 28I and 28J show optical images and schematic illustrations of the tail and liver hemostatic models respectively.

FIG. 29A shows optical images of the performed hematoxylin and eosin (H&E) staining and Masson's trichrome (MT) staining of the wound of diabetic rats with three different wound dressings after 14 days treatment. FIGS. 29B to 29D show epidermal thickness, length of wound area after 6 days and 14 days treatment, and re-epithelialization of wounds with three different wound dressings after 14 days of quantitative analysis of the H&E staining; FIS. 29E to 29G show appendage count, scar elevation index and collagen density of wounds with three different wound dressings after 14 days treatment of quantitative analysis of the MT staining; FIGS. 29H and 29I show optical images IHC staining of the wounds with three different wound dressings after 6 days treatment; FIGS. 29J and 29K show quantitative analysis of the IHC staining.

### DETAILED DESCRIPTION

In the following description, details of the present invention are set forth as preferred embodiments. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

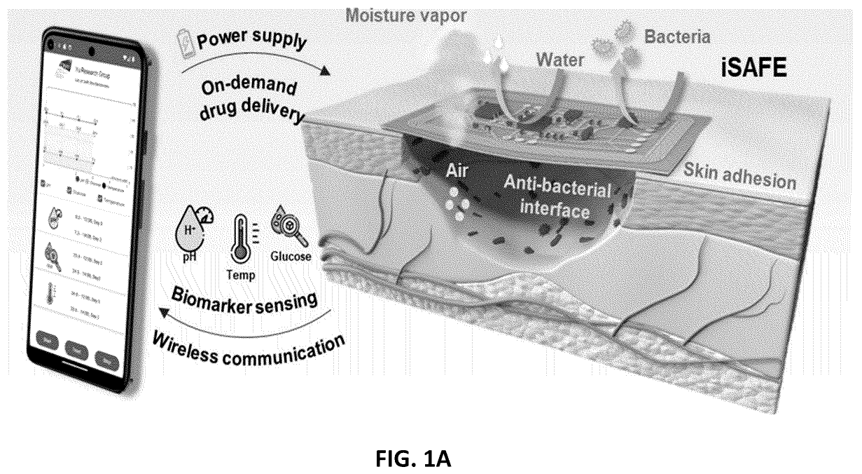

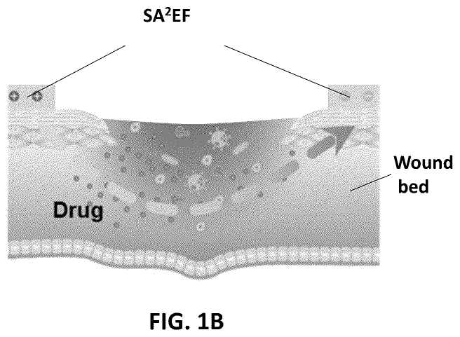

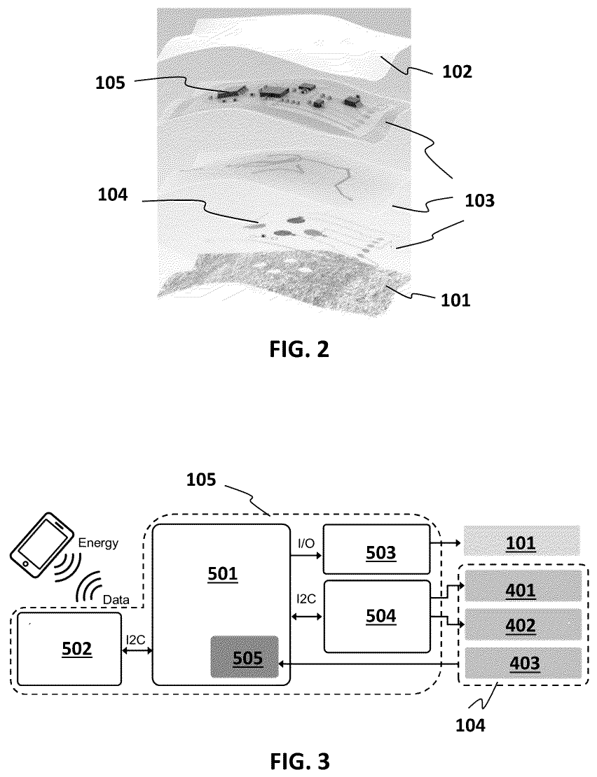

FIG. 1A shows a schematic diagram of an intelligent wound management (iSAFE) system (or intelligent adhesive bandage) in accordance with one embodiment of the present invention being adhered to a skin and operated by a remote device; and FIG. 1B shows a schematic diagram illustrating how drug is released by the intelligent wound management system to a wound bed. FIG. 2 is a block diagram illustrating key components of the intelligent wound management system. FIG. 3 shows an exploded-view illustration of the layered-structure of the intelligent wound management system.

The intelligent wound management system is characterized by an ultra-thin, high-adhesion, multi-layered stack structure, including a bioelectrical interface layer 101; a waterproof moisture-permeable protective film 102; and a permeable stretchable circuit assembly 103 arranged between the bioelectrical interface film 101 and the protective film 102.

In some embodiments, the bioelectrical interface film 101 is a stretchable adhesive antibacterial film made by co-electrospinning of styrene-ethylene-butylene-styrene (SEBS) fiber and gelatin methacrylate (GelMA) fiber loaded with antibiotics and electrically conductive nanoparticles. Preferably, the stretchable adhesive antibacterial film contains hydrogen bonding crosslinking amine and hydroxyl groups in polymer chains of the GelMA. Preferably the weight ratio of SEBS to GelMA is equal to 1:1 to have a balanced mechanical and biological performance. Preferably, the electrically conductive nanoparticles may be, but not limited to, silver, gold, or platinum nanoparticles.

The permeable stretchable circuit assembly 103 comprises a permeable stretchable circuit board; and one or more biosensors 104 constructed on the permeable stretchable circuit board. In some embodiments, the one or more biosensors may include a glucose sensor 401, a pH sensor 402 and a thermal sensor 403 for in-situ monitoring of glucose, pH, and/or temperature (Tem) of wound beds, providing critical insights into the wound conditions.

The permeable stretchable circuit assembly 103 further comprises electronic components 105 assembled on the permeable stretchable circuit board 103. Key electronic components 105 of the intelligent wound management system include a microcontroller unit (MCU) 501, a memory (not shown), a wireless energy harvesting and communication module 502, a drug delivery actuation module 503 electrically coupled to the bioelectrical interface film for adaptive drug delivery for wound treatment, and a physiological signal processing module 504 electrically coupled to one or more biosensors 104 for in-situ wound monitoring.

The MCU 501 may be in communication with the memory and configured for processing and storing data acquired from the biosensors 104. The energy harvesting and communication module 502 may include an NFC circuit chip for achieving battery-free wireless energy and data transmission, allowing the entire circuit to be activated simultaneously and facilitating remote diagnosis and treatment of wound when a smartphone is brought close to the intelligent wound management system.

The drug delivery actuation module 503 may include at least one pair of drug delivery electrodes and a switch for switching on/off a drug delivering voltage applied on the bioelectrical interface film 101 through the drug delivery electrodes for adaptive treatment and drug delivery. In some embodiments, the switch may be a semiconductor switching device, such as, but not limited to be, a N-type MOSFET (BSS138BKVL, Nexperia).

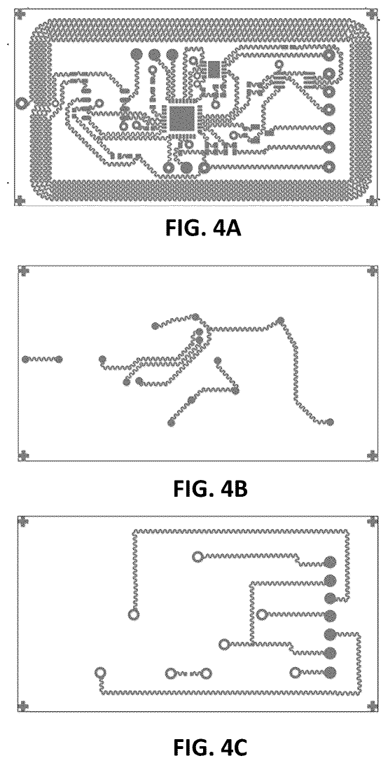

FIGS. 4A to 4C shows layouts of stretchable conductive trace layers for the permeable stretchable circuit board in accordance with one embodiment. The first and second trace layouts are trace for connecting and routing the battery-free functional circuits for intelligent wound monitoring and therapy. The third trace layout is the trace for connecting and routing the drug delivery electrodes, sensor electrodes and electronic components. The overall circuit board is small with a size of 51.69 mm×28.05 mm.

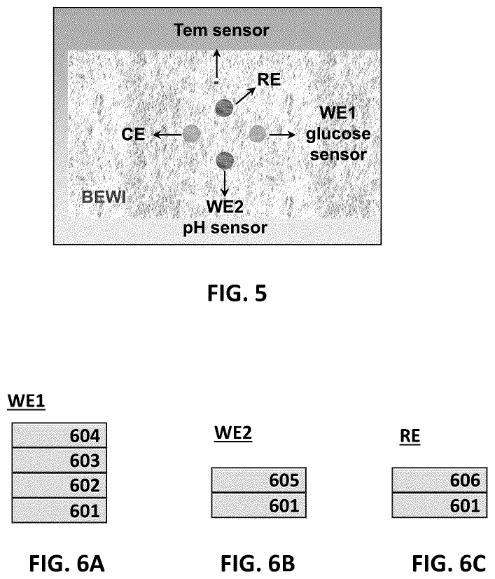

Referring to FIG. 5, the glucose sensor may include a working electrode (WE1), a counter electrode (CE) and a reference electrode (RE), configured to generate current outputs related to glucose concentration in the wound bed. The pH sensor may include a working electrode (WE2) and share the reference electrode with the glucose sensor, and configured to generate a voltage output related to pH values of the wound bed; and thermal sensor may be implemented with a thermal resistor (e.g., NCP15WF104F03RC).

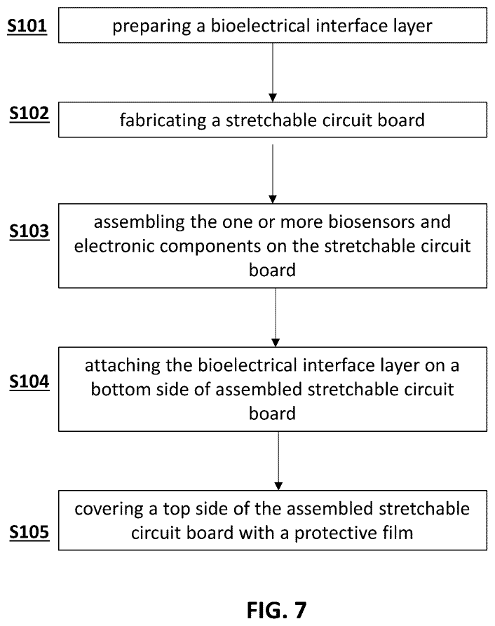

Referring to FIG. 6A, the working electrode (WE1) of the glucose sensor may have a layered-structure including: a stretchable base 601; a mediator layer 602 deposited on the stretchable base 601; a layer of glucose sensing element 603 deposited on the mediator layer 602; and a layer of entrapping material 604 deposited on the layer of glucose sensing element 603. Referring to FIG. 6B, the working electrode (WE2) of the pH sensor may have a layered-structure including: a stretchable base 601; and a layer of pH sensing element 605 deposited on the stretchable base 601. The reference electrode may have a stretchable layered-structure including: a stretchable base 601; and a layer of Ag/AgCl 606 deposited on the stretchable base 601.

In some embodiments, the stretchable base electrode 601 may be made of any suitable types of stretchable materials, such as, but not limited to be, SEBS, with coating of conductive materials such as gold, silver, copper or platinum. The mediator layer 602 may be made of any suitable types of mediator materials applicable for implantable devices, such as, but not limited to be, Prussian blue, carbon nanomaterials (like graphene, carbon nanotubes). The glucose sensing element 603 may be a glucose oxidase or glucose dehydrogenase. The entrapping material 604 may be a Chitosan or any suitable kind of entrapping materials applicable to implantable devices. The pH sensing element 605 may be made of IrOₓ, polyaniline, or any suitable types of pH sensing elements with excellent biocompatibility for implantable or clinical applications.

The measurements of glucose concentration, pH value, and temperature are performed based on a potentiostatic method, open-circuit voltage (OCV) method, and voltage division method, respectively. More specifically, the physiological signal processing module 504 may include a potentiostat, which is a three-electrode electrochemical interface for enabling rapid detection of glucose potency.

In one embodiment, the potentiostat interface (or physiological signal processing module) 504 may be constructed using a dual op-amplifier (e.g., AD8606, Analog Device) and a digital-to-analog converter (DAC) (e.g., MCP47CVB12-E/MF, Microchip Technology). The dual op-amplifier was used to achieve the three-electrode system performance and current-to-voltage conversion. Meanwhile, the DAC enables differential pulse voltammetry (DPV) dynamic excitation signal to bias the reference and working electrodes. The analog signals from the sensors are then converted into digital signals through the analog-to-digital converter (ADC) 505 inside the MCU 501. When the sensor results were delivered from the MCU via the inter-integrated circuit, the NFC chip could transfer the data to the smartphone via radio frequency (RF) interface when the smartphone is brought in proximity.

Referring back to FIG. 1B, when infection occurs and is detected by the biosensors, the anti-infection drug (like antibiotics) contained in the intelligent wound management system would be released to the wound bed by applying voltage to the bioelectrical interface through the drug delivery electrodes. The drug release/delivery amount may be adjusted by (the MCU) by controlling a product of the applied voltage and the period of switching on the applied voltage, while the amount can be negligible when no voltage is applied. In this way, the released amount could be intelligently controlled by the wound conditions assessed by biosensor and thus prevent the overuse and abuse of drugs.

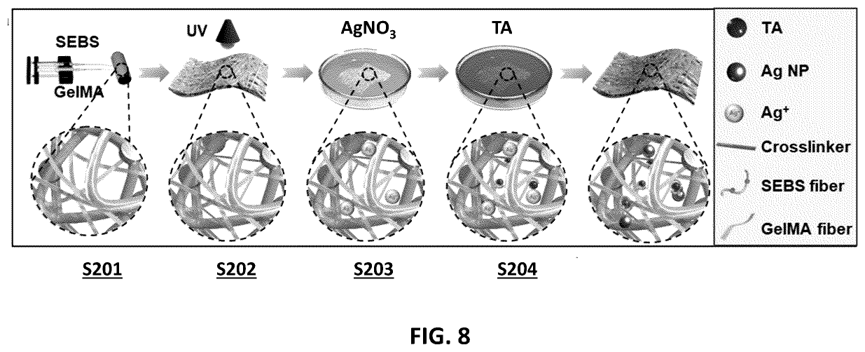

FIG. 7 illustrate a flowchart of a method S100 for fabricating the intelligent wound management system in accordance with one embodiment of the present invention. The method S100 includes:
S101: preparing a bioelectrical interface film;
S102: fabricating a stretchable circuit assembly;
S103: attaching the bioelectrical interface film on a bottom side of the stretchable circuit assembly; and
S105: covering a top side of the stretchable circuit assembly with a protective film.

Several holes corresponding to positions of working electrodes of sensors (as shown in FIG. 5) are formed (e.g., by laser-cutting) on the bioelectrical interface film 101 for exposing the working electrodes such that the working electrodes can be in direct contact with wound exudate without negative impacts on the functions of SA²EF. The holes may be formed on the bioelectrical interface film before the bioelectrical interface film being attached to the stretchable circuit assembly.

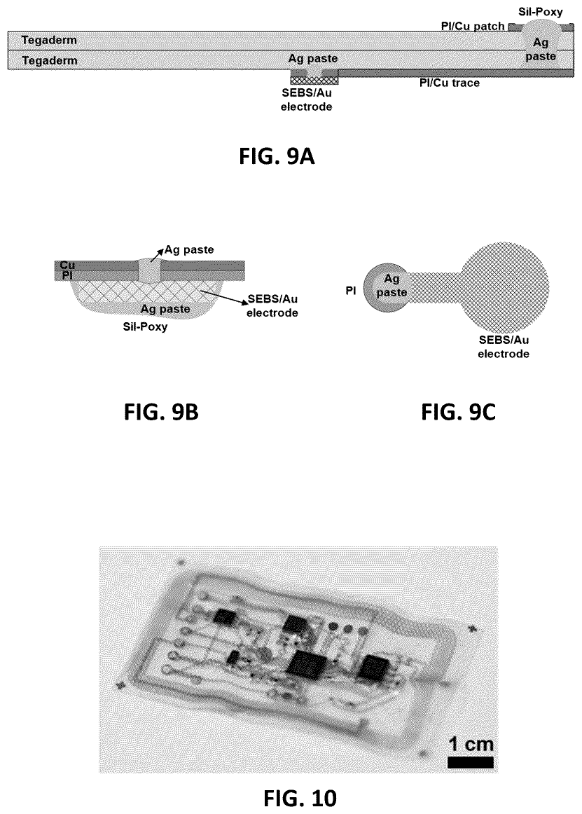

FIG. 8 shows a schematic diagram illustrate a fabrication process flow for the bioelectrical interface film in accordance with one embodiment of the present invention. As shown, the fabrication process includes the steps of:

S201: preparing a first syringe containing a SEBS precursor solution and a second syringe containing a drug-loaded GelMA solution; connecting the first and second syringes to a dual-channel syringe pump to eject out SEBS fiber and GelMA fiber simultaneously; and co-electrospinning the SEBS fiber and GelMA fiber on a metallic foil covering on a rotating drum to obtain a fibrous film composed of SEBS and GelMA fibers;

S202: immersing the fibrous film in a solution of 2-Hydroxy-4′-(2-hydroxyethoxy)-2-methylpropiophenone (photoinitiator 2959, 8 wt. %) under UV exposure crosslink the GelMA fiber; and then peeling off the fibrous film from the metallic foil;

S203: immersing the S/G fibrous film in a silver nanoparticle solution (e.g. Ag NO3) to load silver nanoparticle to the cured S/G fibrous film;

S204: immersing the silver-nanoparticle-loaded S/G fibrous film in a polyphenolic solution (e.g. tannic acid (TA)) to form the bioelectrical interface film, wherein the bioelectrical interface film is modified with tannic acid to contain catechol and pyrogallol, and adherable to the skin through multiple synergistic reactions including hydrogen bonding and Schiff base/Michael addition reactions, electrostatic attraction, and cation-π interactions.

More specifically, the SEBS precursor solution is obtained by solving SEBS (80 wt. %) and F127 (20 wt. %) in chloroform (80 wt. %) and toluene (20 wt. %). The drug-loaded GelMA solution is a mixed GelMA/antibiotics solution having 10 wt. % GelMA and 0.2 wt. % penicillin sodium in hexafluoroisopropanol.

Both syringes have capacities of 20-mL and are equipped with 21 G metal nozzles. The SEBS fiber and GelMA fiber are ejected out of the syringes at flow rates of 0.8 ml/h and 1.0 ml/h respectively. The nozzles of both syringes are set to have a lateral (x-axial) sliding speed between 10 to 12 mm/s to ensure a uniform distribution of the fibrous mat.

For co-electrospinning the SEBS and GelMA fibers, positive electrodes are attached to nozzles of the two syringes to apply voltages ranging from 18 to 23 kV to the SEBS precursor solution and the drug-loaded GelMA solution respectively. The metallic foil is an aluminum foil. The rotating drum has a 10 cm diameter and is set to rotate at 90 to 100 revolutions per minute and positioned 15 cm from the nozzle tips. The entire electro-spinning process is conducted over 12 hours.

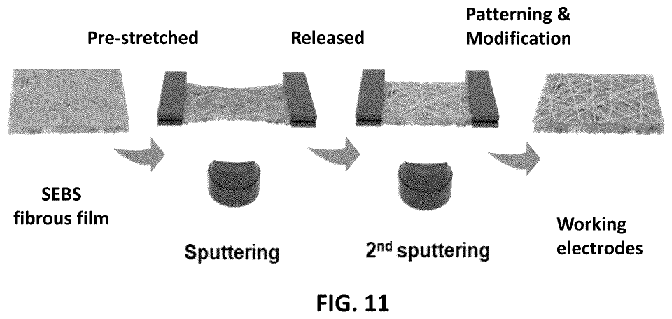

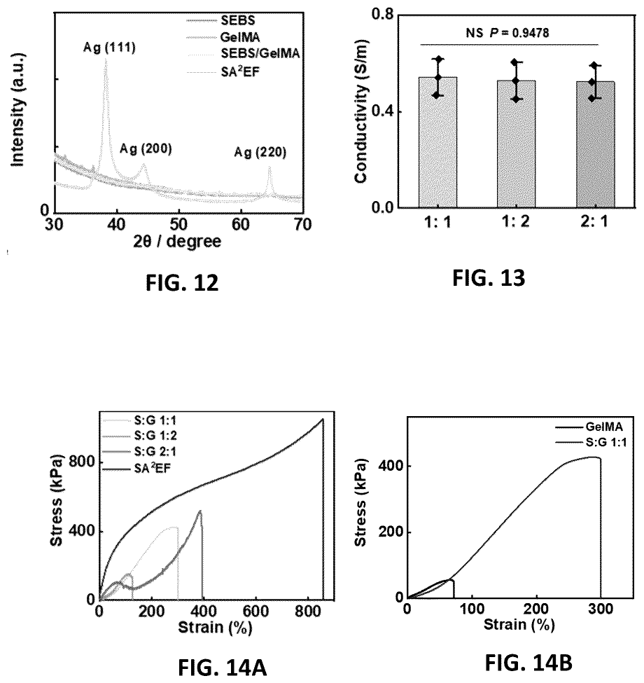

FIG. 9A shows a frontal plane of the vertical integration approach for fabricating the stretchable circuit assembly. FIGS. 9B and 9C show a frontal plane and bottom view of the connection points between Cu traces and sensor electrodes. The three layers of circuit and electrodes are vertically connected by advanced fabrication techniques for highly-integrated, small-sized and ultrathin epidermal electronics with permeable and waterproof encapsulation, resulting in a stretchable circuit assembly (FIG. 10) with excellent flexibility and an ultrathin thickness of 140 μm.

More specifically, the fabrication process for the stretchable circuit assembly may include the following steps:

S301: forming a first Tegaderm/Cu trace layer with a first Cu trace layout (e.g., the Cu trace layout shown in FIG. 4A)

S302: forming a second Tegaderm/Cu trace layer with a second Cu trace layout (e.g., the Cu trace layout shown in FIG. 4B)

S303: bonding the first Tegaderm/Cu trace layer and the second

Tegaderm/Cu trace layer to form a stretchable circuit board having a first Cu trace on its top surface and a second Cu trace on its bottom surface (FIG. 9A); and forming connection holes on the stretchable circuit board to provide electrical interconnects between the first Cu trace and the second Cu trace; wherein the connection holes may be formed by laser-cutting, filling the laser-cut holes with Ag paste; and applying a protective layer of Sil-Poxy on the filled Ag paste;

S304: depositing a PI/Cu sheet on a water solution tape, laser-cutting the PI/Cu sheet to form a third conductive trace and patching holes; filling Ag paste into the patching holes for the later connecting between Cu trace and sensors; applying a protective layer of Sil-Poxy on the filled Ag paste;

S305: transfer-printing the third conductive trace to the bottom surface of the stretchable circuit board;

S306: bonding and electrically connecting (e.g., by low temperature solder joints) stretchable sensor electrodes (FIGS. 9B and 9C) and electronic components on the stretchable circuit board; and attaching an encapsulating layer of Tegaderm on top of the electronic components.

The fabrication process for each Tegaderm/Cu trace layer may include the following steps:
S401: attaching a layer of Tegaderm on a supporting substrate;
S402: depositing a PI/Cu sheet on the first layer of Tegaderm;
S403: patterning the PI/Cu sheet through photolithography and wet etching to obtain the Tegaderm/Cu trace layer.

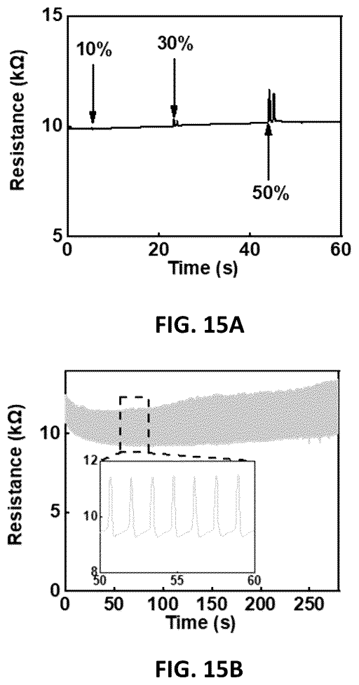

The stretchable sensors electrodes are prepared by using a pre-stretching strategy on electrospun SEBS fibrous film to ensure the electrodes to be conductive even under the stretching state. FIG. 11 shows a schematic diagram for a fabrication process of stretchable electrodes for subsequent construction of biosensors. Firstly, the electrospun SEBS fibrous film is pre-stretched (e.g., for 50%) and then deposited with a first layer of Cr (10 nm)/Au (100 nm) by sputtering. Next, the pre-stretched film is released and then deposited with a second layer of Cr/Au (on top of the first layer of Cr/Au) to form a stretchable SEBS/Au layer. Then, the SEBS/Au layer is patterned by laser-cutting to form stretchable SEBS/Au electrodes.

The modification of corresponding SEBS/Au electrodes to the working electrode for glucose biosensor includes the following steps:
S501: electrodepositing a layer of Prussian blue on the corresponding SEBS/Au electrodes by cyclic voltammetry from 0 V to 0.5 V for 5 cycles in the mixed solution of 2.5 mM ferric chloride (FeCl₃), 100 mM potassium chloride (KCl), 2.5 mM Potassium ferricyanide (K₃Fe(CN)₆), and 100 mM hydrogen chloride (HCl);
S502: dipping a cocktail of 2 μL GOx (5U/μl), 1 μL bovine serum albumin (BSA, 2 mg/mL) and 2 μL glutaraldehyde (GA, 2%) on the electrodes;
S503: storing the electrodes in the 4° C. refrigerator for overnight; and
S504: dropping 2 μL Chitosan (2%) on the electrodes for immobilizing enzymes.

The modification of corresponding SEBS/Au electrode to the working electrode for the pH sensor includes electrodepositing IrOₓ on the SEBS/Au electrodes under a constant voltage of 0.7 V for 45 minutes in an electrodeposition electrolyte. The electrodeposition electrolyte is prepared by:
S601: dissolving 300 mg iridium tetrachloride into 200 mL deionized (DI) water to form a exist solution;
S602: adding 2 mL hydrogen peroxide (H₂O₂) into the exist solution with continuous stirring;
S603: adding 1000 mg of oxalic acid dihydrate with continuous stirring;
S604: introducing small quantities of anhydrous potassium carbonate to the continuously stirred solution to adjust the pH value of the solution to 10.5;
S605: storing the pH-adjusted solution at ambient room temperature for 48 hours for stabilization.

The modification of corresponding SEBS/Au electrode to the reference electrode includes injecting 10 μL 0.1 M FeCl₃ on the electrode to obtain a layer of Ag/AgCl on the SEBS/Au electrode.

### Characterization of the Bioelectrical Interface Film (SA²EF)

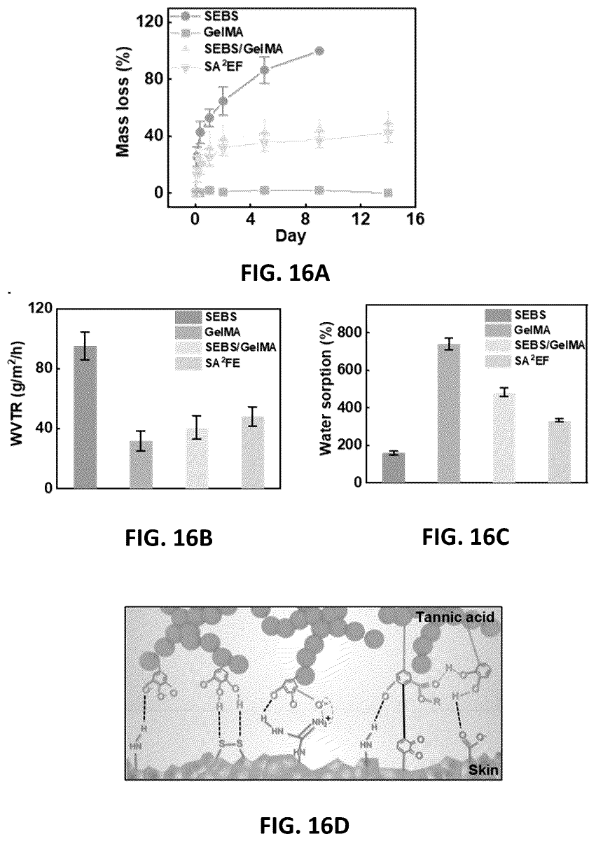

As shown in FIG. 12, X-ray diffraction (XRD) analysis demonstrates the characteristic diffraction peaks at 38.1, 44.3, and 64.6, corresponding to (111), (200), and (220) crystallographic planes, are existed in the SA²EF, indicating successful synthesis of AgNPs mediated by TA.

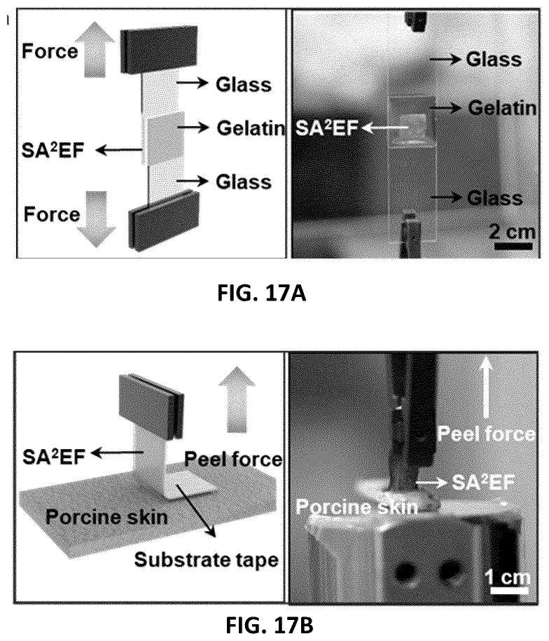

To optimize the performance of the SA²EF, the impact of SEBS and GelMA fiber ratio on the conductivity is explored. As shown in FIG. 13, all the SA²EFs with different ratios of SEBS to GelMA show great conductivity of around 0.52 S/m, demonstrating the conductivity is predominantly dependent on the loading amount of Ag NPs, rather than the SEBS/GelMA (S/G) ratio.

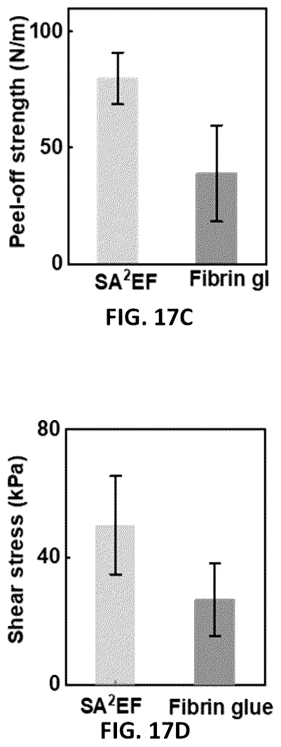

FIGS. 14A and 14Bexhibit the stress-strain curves of GelMA, S/G fibers with different content ratios as well as the SA²EF. It is clear that the co-electrospinning strategy largely improves the stretchability of the fibers and the stretchability is enhanced with the increase of SEBS content. In addition, the mechanical strength and stretchability of SA²EF (SEBS: GelMA 1:1) are further increased because of the enhanced intermolecular interactions by TA.

Tannic acid is a polyphenolic compound that can crosslink the hydroxyl groups in the GelMA polymer chain with the amine groups in the skin through strong hydrogen bonds. Tannic acid contains catechol (catechol) and pyrogallol, which form numerous hydrogen bonds with amine groups (—NH₂), amide groups (—CONH—), and hydroxyl groups (—OH) in skin surface proteins (such as keratin). The amine groups (—NH₂) and hydroxyl groups (—OH) of GelMA can form hydrogen bonds with the phenolic hydroxyl groups of tannic acid (similar to the effect of skin proteins), making tannic acid more stably anchored in the GelMA network. This additional crosslinking via hydrogen bonding results in a denser network structure, leading to improved mechanical properties like higher tensile strength and stretchability.

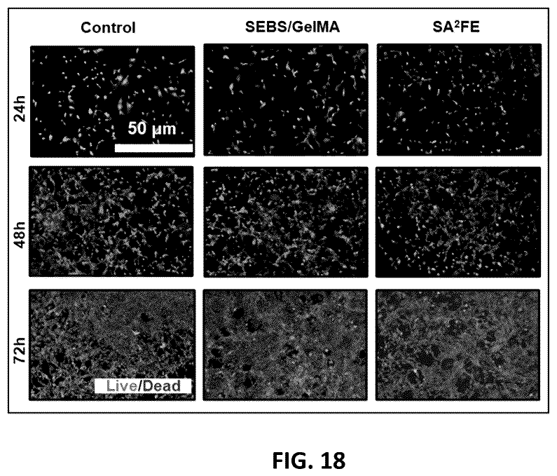

Besides, the resistance responses of the optimized SA²EF under stretching configure are tested. As shown in FIGS. 15A and 15B, the resistance change is negligible under 10% and 30% stretching. Although there are changes of the resistance of the SA²EF when stretched to 50%, the resistance could return to the initial state and the tiny fluctuation is not enough to influence the electrical performance of SA²EF. Additionally, the resistance exhibited stable recovery capabilities even after undergoing in excess of 600 cycles of continuous 50% stretching deformation.

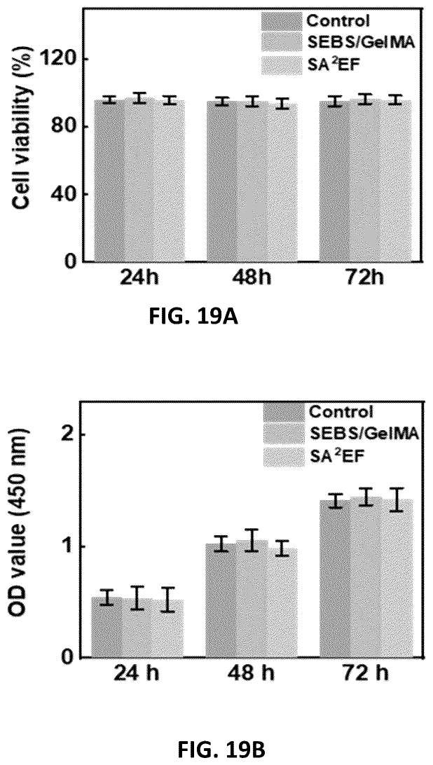

Furthermore, the SA²EF shows appropriate biodegradability (˜40% mass loss during 14-day testing), water vapor transmission rate (WVTR) (˜50 g/m²/h), and swellability (˜300%) (FIGS. 16A to 16C). Due to multiple synergistic interactions including hydrogen bond and Schiff-base/Michael addition reaction, electrostatic attraction, cation-π interaction (FIG. 16D), SA²EF exhibits good tissue adhesiveness, which can conformally attach to skin surface even under twisting and bending.

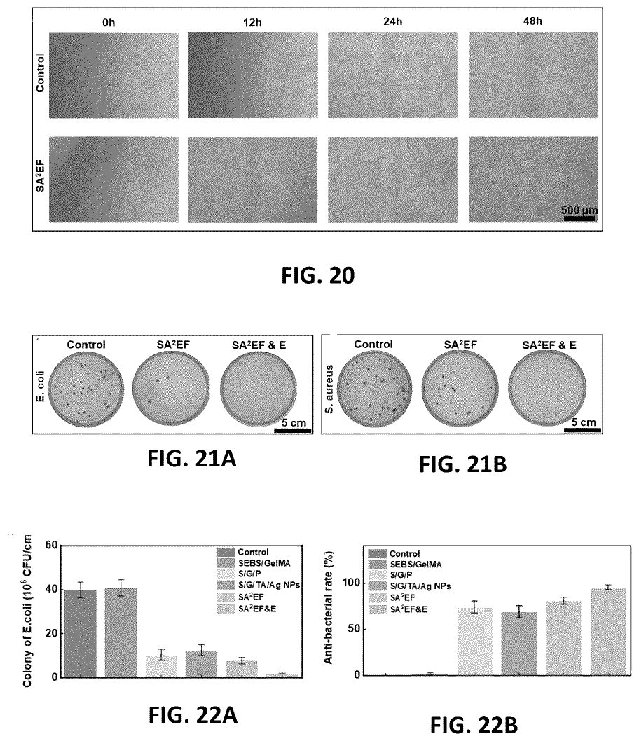

Quatitative analysis shows the shear strength and peel-off strength of SA²EF are much higher than those of commercial fibrin glue, demonstrating its clinical application potential (FIGS. 17A to 17D).

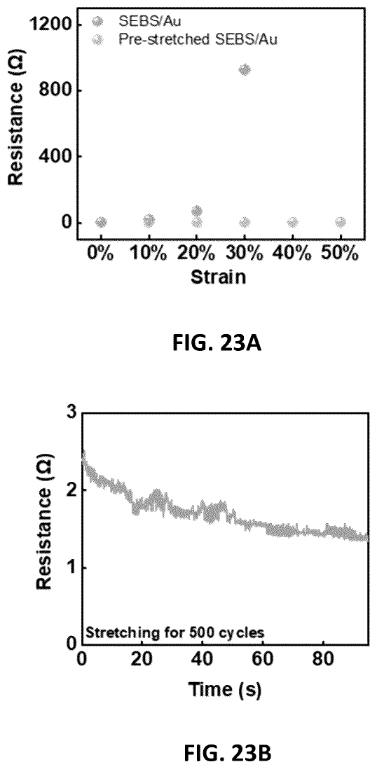

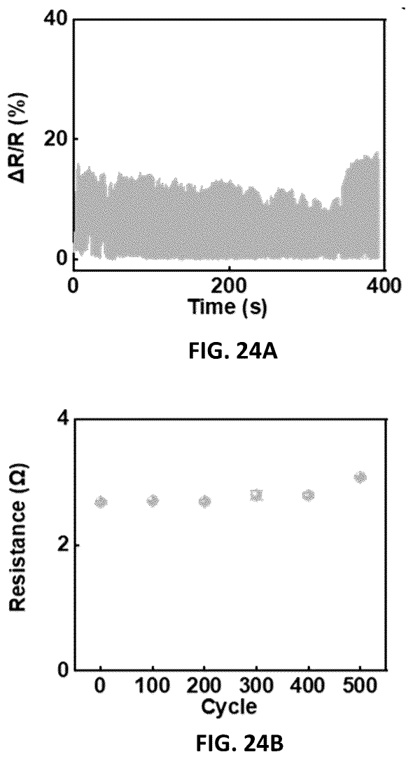

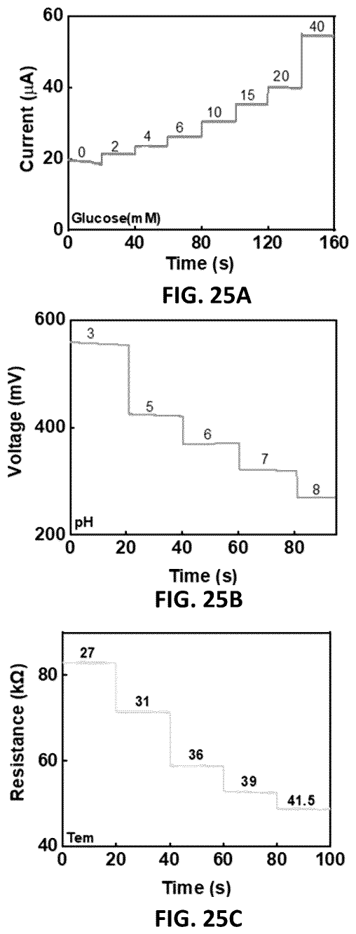

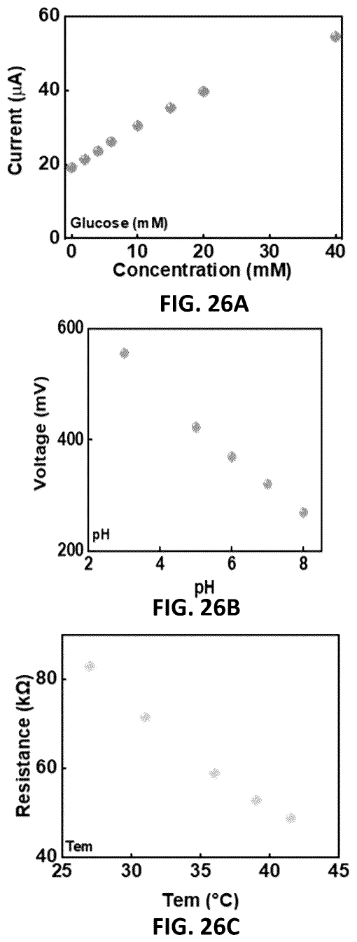

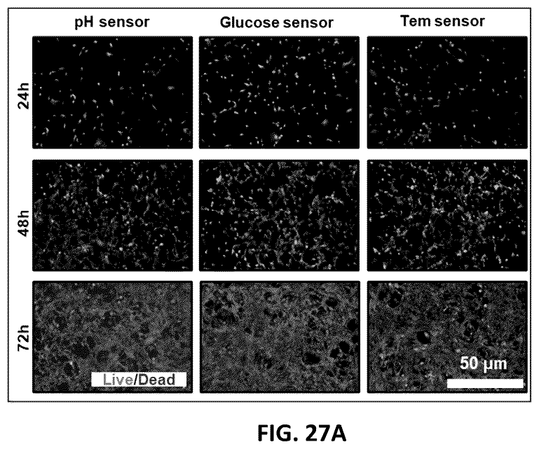

Next, we evaluated the in vitro bioactivity of SA²EF. First, live/dead staining and cell counting kit-8 (CCK-8) assay indicate the favorable biocompatibility of SA²EF, which can facilitate cell survival and proliferation (FIGS. 18, FIGS. 19A and 19B). Wound healing assay, also known as the cell scratch test, further demonstrates SA²EF can promote cell migration and accelerate wound healing rate (FIG. 20). In addition, we find such composite membrane supports cell adhesion and possesses excellent anti-bacterial activity (FIGS. 21A and 21B, FIGS. 22A and 22B), suggesting it can create a beneficial microenvironment for cell growth and shows promising potential as an anti-infective wound dressing.

### Characterization of the Stretchable Biosensors

In the traditional design of stretchable biosensors, the connection between serpentine and electrode patch is always constructed by the island-bridge structure. Although the island-bridge design is able to withstand repeated stretching and bending cycles without the risk of fracture, there is always a larger strain on the site of electrode patch. For highly sensitive biosensors, even minor deformations of the electrode substrate can lead to significant signal fluctuations and potential sensor failure. As the stretchable-conductive electrodes possess superior durability and resistive to physical deformation, skewing their functionality towards high-reliability, even under extensive, dynamic stress conditions, it would be an optional pathway to construct biosensors on stretchable-conductive electrodes for epidermal electronics.

Based on the pre-stretching strategy, the electrospun SEBS fiber could maintain stable conductivity during stretching after two times' sputtering as the conductive fibers are still connected during stretching, enhance the stretchability of the multiple biosensors. The conductivity decrease could be negligible even after 500 cycles continuous stretching and the roles of electrodes would not be influenced.

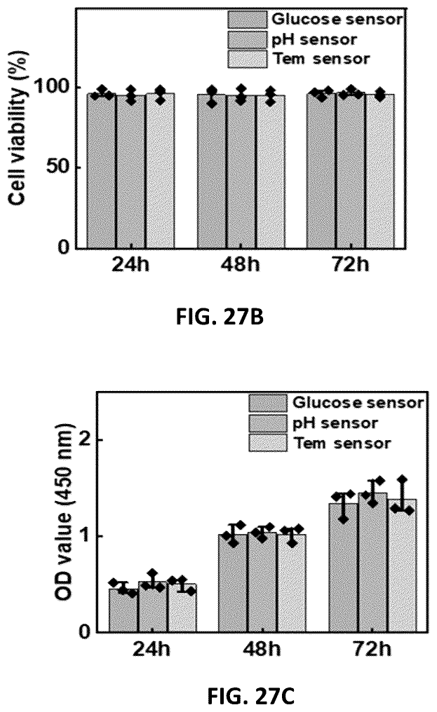

As exhibited in FIGS. 23A and 23B, compared to the normal sputtering SEBS fiber, the pre-stretched SEBS enables to retain stable resistance even under continuous stretching and friction. Although the resistance value would fluctuate during dynamic friction, the conductivity of the sputtered SEBS fiber could maintain relatively stable even after 400 cycles. can ensure a basically stable resistance even under the stretching state. The conductive SEBS fiber film can be laser-cut as electrodes with different shapes and sizes. Collectively, such electrodes possess superior durability and resistance to physical deformation, thereby maintaining high reliability even under extensive and dynamic stress conditions.

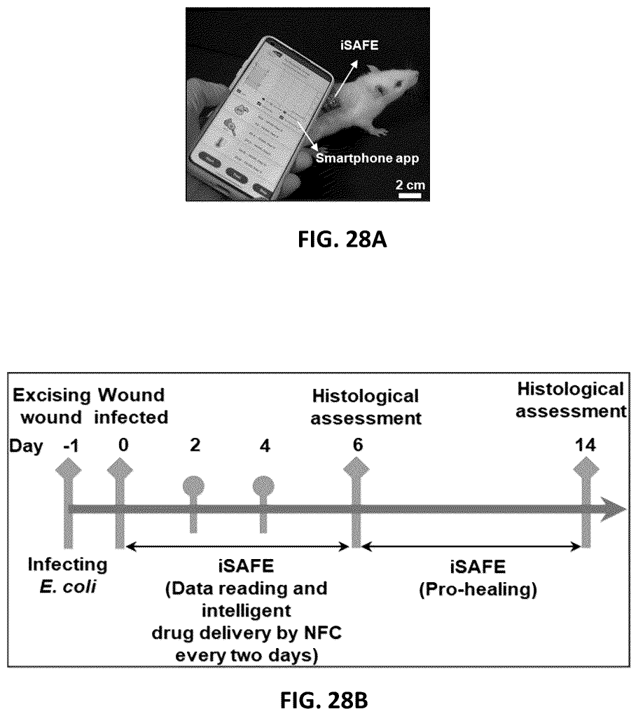

As a result, the glucose sensor and pH sensor modified on the electrode both show great stretchability and stable output under 30% stretching (FIGS. 24A and 24B). Benefiting from the superior performance of sensing elements of GOx, the current outputs of glucose sensor are linearly related with glucose concentration in electrolyte. The coefficient of determination (R²) could reach at IrOₓ of 0.989.

Similarly, the pH sensor modified by IrOₓ could sensitively detect the hydrogen ion changes in electrolyte with a R² of 0.996. Furthermore, the glucose and pH sensors show superior anti-interference ability and stability.

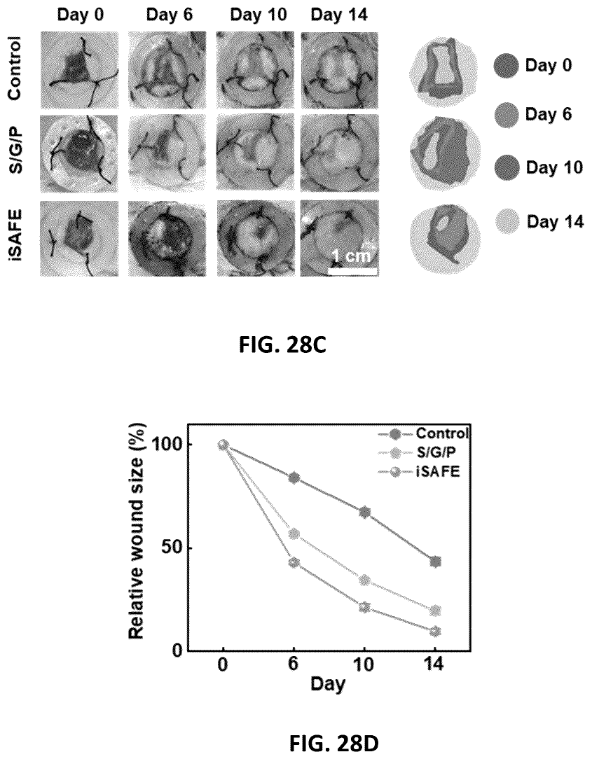

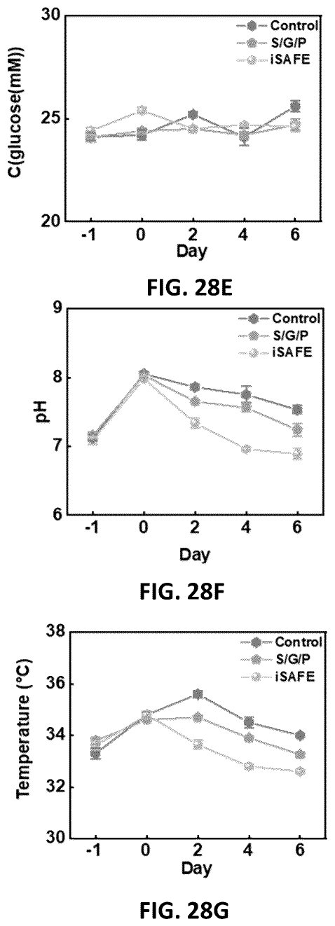

FIGS. 25A to 25C show the responses of the glucose, pH and tem sensors as a function of time, and FIGS. 26A to 26C demonstrate their corresponding calibration curves. As shown in FIGS. 25A and 26A, the current response of the glucose sensor would increase with the increase of glucose concentration in electrolyte under a constant voltage of −0.1 V, and there is great linearity between the glucose concentration in electrolyte and the current output of the glucose sensor.

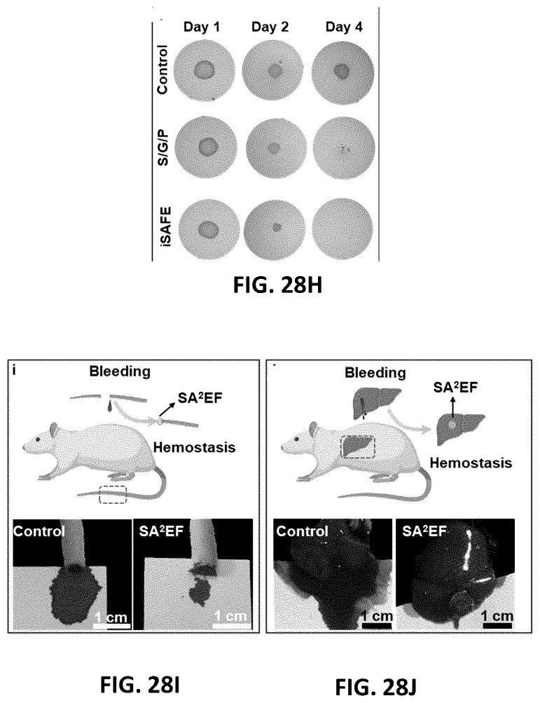

As these sensors are intended for monitoring wound exudate and will be in direct contact with open tissue, biocompatibility is a paramount consideration that must be given primacy. The NIH/3T3 cells co-cultured results, as exhibited in FIGS. 27A to 27C, indicate that there is no obvious cytotoxicity of the sensing elements, electrodes and other components of glucose, pH and Tem sensors. It is worth mentioning that the optimal choice of IrOₓ as the sensing element for the pH sensor is principally attributed to its exceptional biocompatibility compared to cheap polyaniline with risks of cytotoxicity.

### In-Vivo Preclinical Study

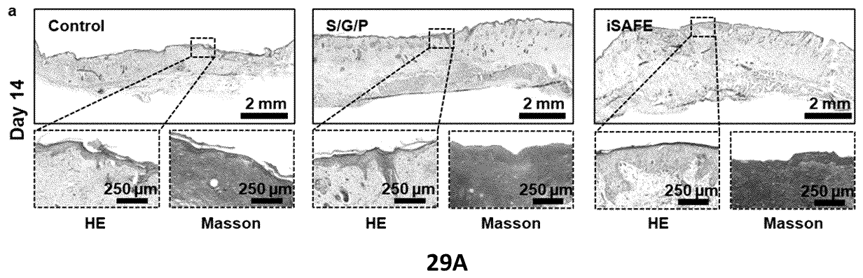

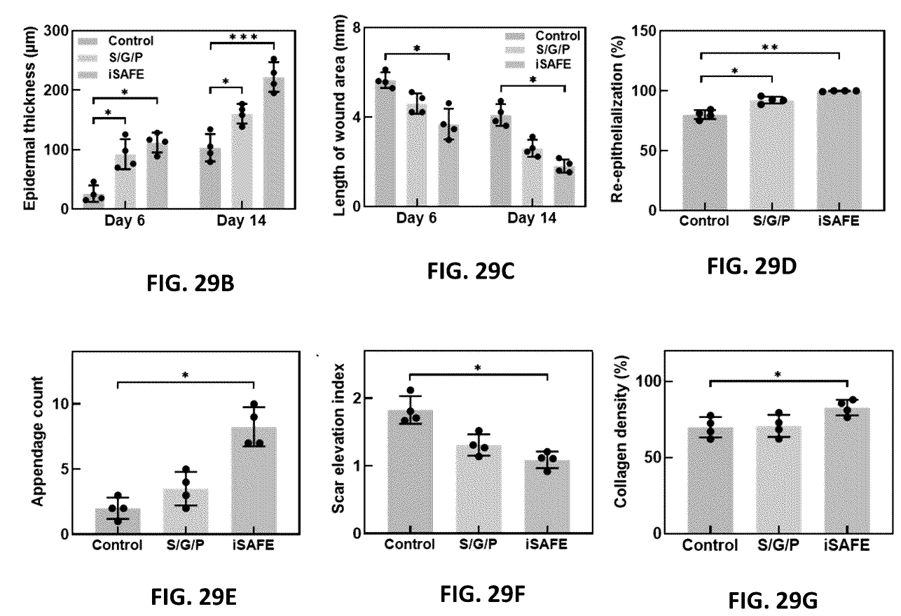

FIGS. 28A to 28J show the in-vivo preclinical evaluation on diabetic rats to test the robustness and efficacy of the intelligent wound management system in wound monitoring and accelerating wound healing. As exhibited in FIG. 28A, the intelligent wound management system can be seamlessly attached to the back of the rat and our developed state-of-the-art biosensors are able to monitor wound state by the GUI (FIG. 28A). For the diabetic rats, bilateral full-thickness excisional wounds and infection by E. coli (20 μl, 106 CFU/mL) were made on Day-1 (FIG. 28B). Here, three different groups were tested for verifying the validity of the intelligent wound management system: Control group (Tegaderm dressing without controlled drug releasing), S/G/P group (co-electrospinning SEBS/GelMA/Drug dressing without controlled drug releasing) and the intelligent wound management system group.

From Day 0 to Day 6, we measured the glucose level, pH and Tem of the wound exudate and exchange wound dressings every two days for each group. For the intelligent wound management system group, the drug would be simultaneously delivered if the wound was infected during measuring. In the early stage, the drug treatment and wound dressings play roles in eliminating bacterial infections and modulating immune reactions.

After Day 6, little wound exudate could be found, and thus, the sensing and drug treatments were withdrawn. Wound dressings are employed to accelerate cell migration and wound healing in the next stage. As exhibited in FIGS. 28C and 28D, the highest wound closure rate is observed in the intelligent wound management system group, while that of the S/G/P group is higher the control group, demonstrating that the effectiveness of the intelligent wound management system on the pro-healing. The pro-healing performance of the S/G/P group may be led by the presence of GelMA with drug.

For the glucose level in first 6 days, as shown in FIG. 28E, the glucose levels are stable for all diabetic rats. It could be seen that the overall tendency of the three groups of pH and Tem value decreases (FIGS. 28F and 28G), meaning that the appearance of wound healing. The obvious dropped values in the intelligent wound management system group compared to other groups demonstrate the excellent ability of the intelligent wound management system to antibacterial and pro-healing.

Moreover, the mixed bacteria colonies culture from the wound beds of three different groups onto LB agar plates showed significant decrease in bacteria growth in the intelligent wound management system group as compared to other two groups, proving the excellent antibacterial properties of the SA²EF and the intelligent wound management system (FIG. 28H).

We further evaluated the hemostasis of the SA²EF by the tail and liver hemostatic models. As shown in FIGS. 281 and 28J, compared to the control group (without the SA²EF), the blood losses in the SA²EF groups are much lower no matter for the tail or liver hemostatic models, revealing the great hemostasis of the SA²EF.

### Evaluation of Healing Effectiveness

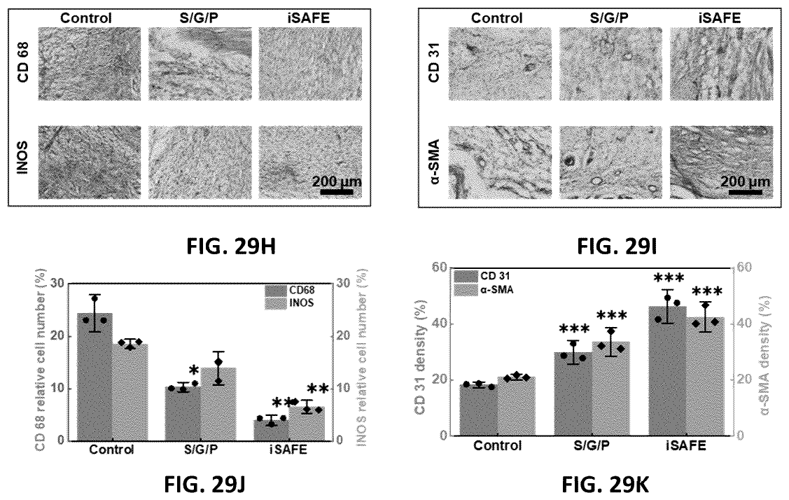

To evaluate the healing effectiveness of the three group wound dressings, we further performed hematoxylin and eosin (H&E), Masson's trichrome (MT) staining and immunocytochemistry analyses of the wound after 6 days and 14 days, respectively. On the 14th day, the wound is almost healed in the intelligent wound management system group while partial tissue defects could be seen in other two groups (FIG. 29A). Mature epithelium, higher epidermal thickness and re-epithelialization as well as lower length of wound area were observed in H&E staining results of the intelligent wound management system group (FIGS. 29B29 to 29D). For the quantitative analysis of H&E staining on the 6th day, it is obvious that the wound indexes of epidermal thickness the intelligent wound management system group are largely higher than those of the control and S/G/P groups while the length of wound area of the intelligent wound management system group is less than the other two groups (FIGS. 29B and 29C). The MT staining images and quantitative analysis results (FIG. 29A and FIGS. 29E to 29G) exhibit a significantly higher dermal appendage count and collagen density with lower scar elevation index for the intelligent wound management system group compared to other two groups, demonstrating the granulation tissue formation and uniform dermis repair. The immunohistochemistry staining (IHC) analyses (FIGS. 29H and 291) and quantitative analysis results (FIGS. 29J and 29K) further show the abilities of antibacterial, accelerate cell migration and promoting wound healing.

The functional units and modules of the [apparatuses, devices, systems, compounds, materials, and/or methods] in accordance with the embodiments disclosed herein may be implemented using computing devices, computer processors, or electronic circuitries including but not limited to application specific integrated circuits (ASIC), field programmable gate arrays ((FPGA), microcontrollers, and other programmable logic devices configured or programmed according to the teachings of the present disclosure. Computer instructions or software codes running in the computing devices, computer processors, or programmable logic devices can readily be prepared by practitioners skilled in the software or electronic art based on the teachings of the present disclosure.

All or portions of the methods in accordance to the embodiments may be executed in one or more computing devices including server computers, personal computers, laptop computers, mobile computing devices such as smartphones and tablet computers.

The embodiments may include computer storage media, transient and non-transient memory devices having computer instructions or software codes stored therein, which can be used to program or configure the computing devices, computer processors, or electronic circuitries to perform any of the processes of the present invention. The storage media, transient and non-transient memory devices can include, but are not limited to, floppy disks, optical discs, Blu-ray Disc, DVD, CD-ROMs, and magneto-optical disks, ROMs, RAMs, flash memory devices, or any type of media or devices suitable for storing instructions, codes, and/or data.

Each of the functional units and modules in accordance with various embodiments also may be implemented in distributed computing environments and/or Cloud computing environments, wherein the whole or portions of machine instructions are executed in distributed fashion by one or more processing devices interconnected by a communication network, such as an intranet, Wide Area Network (WAN), Local Area Network (LAN), the Internet, and other forms of data transmission medium.

While the present disclosure has been described and illustrated with reference to specific embodiments thereof, these descriptions and illustrations are not limiting. The illustrations may not necessarily be drawn to scale. There may be distinctions between the artistic renditions in the present disclosure and the actual apparatus due to manufacturing processes and tolerances. There may be other embodiments of the present disclosure which are not specifically illustrated. Modifications may be made to adapt a particular situation, material, composition of matter, method, or process to the objective and scope of the present disclosure. All such modifications are intended to be within the scope of the claims appended hereto. While the methods disclosed herein have been described with reference to particular operations performed in a particular order, it will be understood that these operations may be combined, sub-divided, or re-ordered to form an equivalent method without departing from the teachings of the present disclosure. Accordingly, unless specifically indicated herein, the order and grouping of the operations are not limitations.

What is claimed is:

1. A skin-adhesive air-permeable intelligent bandage, comprising:
a stretchable adhesive antibacterial bioelectrical interface film made of stretchable adhesive antibacterial fibres;
a waterproof moisture-permeable protective film for protecting the wound from external contaminants; and
a permeable stretchable circuit assembly arranged between the bioelectrical interface film and the protective film;
wherein the permeable stretchable circuit assembly comprises:
a permeable stretchable circuit board;
one or more biosensors constructed on the permeable stretchable circuit board; and
electronic components assembled on the permeable stretchable circuit board and including:
a physiological signal processing module electrically coupled to the one or more biosensors for in-situ wound monitoring; and
a drug delivery actuation module electrically coupled to the bioelectrical interface film for adaptive drug delivery for wound treatment.

2. The skin-adhesive air-permeable intelligent bandage of claim 1, wherein the stretchable adhesive antibacterial bioelectrical interface film is a fibrous film made by co-electrospinning of styrene-ethylene-butylene-styrene (SEBS) fibers and gelatin methacrylate (GelMA) fibers loaded with antibiotics.

3. The skin-adhesive air-permeable intelligent bandage of claim 1, wherein the weight ratio of the SEBS fibers to the GelMA fibers is equal to 1:1.

4. The skin-adhesive air-permeable intelligent bandage of claim 1, wherein the bioelectrical interface film is modified with tannic acid to contain catechol and pyrogallol, and adherable to the skin through multiple synergistic reactions including hydrogen bonding and Schiff base/Michael addition reactions, electrostatic attraction, and cation-π interactions.

5. The skin-adhesive air-permeable intelligent bandage of claim 1, wherein the fibrous film is loaded with electrically conductive nanoparticles.

6. The skin-adhesive air-permeable intelligent bandage of claim 5, wherein the electrically conductive nanoparticles are silver nanoparticles.

7. The skin-adhesive air-permeable intelligent bandage of claim 1, further comprising a wireless energy harvesting and communication module for enabling the skin-adhesive air-permeable intelligent bandage to be in communication with a remote device to facilitate battery-free remote diagnosis and treatment of wound.

8. The skin-adhesive air-permeable intelligent bandage of claim 1, wherein the drug delivery actuation module includes: at least one pair of drug delivery electrodes for applying a drug delivering voltage on the bioelectrical interface film; and a switch connected to the drug delivery electrodes and configured for switching on/off the drug delivering voltage.

9. The skin-adhesive air-permeable intelligent bandage of claim 8, further comprising a microcontroller unit electrically connected to the drug delivery actuation module and configured to adjust drug delivery amount by controlling a period of switching on the drug delivering voltage.

10. The skin-adhesive air-permeable intelligent bandage of claim 1, wherein
the one or more biosensors include a glucose sensor having at least one stretchable working electrode attached to the permeable stretchable circuit board; and
the working electrode has a layered-structure including: a stretchable base; a mediator layer deposited on the stretchable base; a layer of glucose sensing element deposited on the mediator layer; and a layer of entrapping material deposited on the layer of glucose sensing element.

11. The skin-adhesive air-permeable intelligent bandage of claim 1, wherein
the one or more biosensors include a pH sensor having least one stretchable working electrode attached to the permeable stretchable circuit board; and
the working electrode has a layered-structure including: a stretchable base; and a layer of pH sensing element deposited on the stretchable base.

12. The skin-adhesive air-permeable intelligent bandage of claim 1, wherein the one or more biosensors include a thermal sensor attached on the permeable stretchable circuit board.

13. A method for manufacturing the skin-adhesive air-permeable intelligent bandage of claim 1, comprising:
fabricating the bioelectrical interface film;
fabricating the stretchable circuit assembly;
attaching the bioelectrical interface film on a bottom side of stretchable circuit assembly; and
covering a top side of the stretchable circuit assembly with a protective film.

14. The method of claim 13, wherein the bioelectrical interface film is fabricated by:
preparing a first syringe containing a SEBS precursor solution and a second syringe containing a drug-loaded GelMA solution;
connecting the first and second syringes to a dual-channel syringe pump to eject out SEBS fiber and GelMA fiber simultaneously;
co-electrospinning the SEBS fiber and GelMA fiber on a metallic foil covering on a rotating drum to form a fibrous film composed of SEBS and GelMA fibers;
immersing the fibrous film in a solution of 2-Hydroxy-4′-(2-hydroxyethoxy)-2-methylpropiophenone under UV exposure to crosslink the GelMA fiber in the fibrous film;
peeling off the fibrous film from the metallic foil;
immersing the fibrous film in a silver nanoparticle solution to load silver nanoparticles to the fibrous film; and
immersing the fibrous film in a tannic acid to form the bioelectrical interface film;
wherein the bioelectrical interface film is modified with the tannic acid to contain catechol and pyrogallol, and adherable to the skin through multiple synergistic reactions including hydrogen bonding and Schiff base/Michael addition reactions, electrostatic attraction, and cation-π interactions.

15. The method of claim 13, wherein the stretchable circuit assembly is fabricated by:
forming stretchable SEBS/Au electrodes;
forming sensor electrodes based on the stretchable SEBS/Au electrodes; and
bonding the sensor electrodes and electronic components on the stretchable circuit board;
wherein the stretchable SEBS/Au electrodes are formed by:
pre-stretching a SEBS film;
depositing a first layer of Cr/Au on the SEBS film;
releasing the SEBS film;
depositing a second layer of Cr/Au on the first layer of Cr/Au to form a stretchable SEBS/Au layer; and
patterning the stretchable SEBS/Au layer to form the stretchable SEBS/Au electrodes.
